SEQUENCE LISTING (15 sequences)

SEQ ID NO: 1 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 2 - protein, 122 aa, Homo sapiens
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 3 - DNA, 321 bp, Homo sapiens
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgacgttcggccaagggaccaaggtggaaatcaaa

SEQ ID NO: 4 - protein, 107 aa, Homo sapiens [sequence omitted]

SEQ ID NO: 5 - protein, 11 aa, Homo sapiens
RASQSVSSYLA

SEQ ID NO: 6 - protein, 7 aa, Homo sapiens
DASNRAT

SEQ ID NO: 7 - protein, 10 aa, Homo sapiens
QQRSNWPPTF

SEQ ID NO: 8 - DNA, 372 bp, Homo sapiens
  source 1..372: mol_type = other DNA
gaggtgcagctgttggagtctgggggaggcttggtacagcctggggggtccctgagactctcatgtgcagtctctggattcacctttaacagctttgccatgagctgggtccgccaggctccagggaaggggctggagtgggtctcagctattagtggtagtggtggtggcacatactacgcagactccgtgaagggccggttcaccatctccagagacaattccaagaacacgctgtatctgcaaatgaacagcctgagagccgaggacacggccgtatatttctgtgcgaaagataagattctctggttcggggagcccgtctttgactactggggccagggaaccctggtcaccgtctcctcagcctcc

SEQ ID NO: 9 - protein, 124 aa, Homo sapiens
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSAS

SEQ ID NO: 10 - protein, 5 aa, Homo sapiens
SFAMS

SEQ ID NO: 11 - protein, 17 aa, Homo sapiens
AISGSGGGTYYADSVKG

SEQ ID NO: 12 - protein, 13 aa, Homo sapiens
DKILWFGEPVFDY

SEQ ID NO: 13 - protein, 107 aa, Homo sapiens
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 14 - protein, 122 aa, Homo sapiens
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 15 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

(19) United States
(12) Patent Application Publication
(10) Pub. No.: US 2023/0201227 A1
(43) Pub. Date: Jun. 29, 2023

(54) COMBINATION THERAPY FOR CANCER TREATMENT

(71) Applicant: Takeda Pharmaceutical Company Limited, Osaka (JP)

(72) Inventors: Richard Labotka, Des Plaines, IL (US); Andrew Fergus, Andover, MA (US)

(21) Appl. No.: 18/184,918

(22) Filed: Mar. 16, 2023

Related U.S. Application Data
(63) Continuation of application No. 17/903,507, filed on Sep. 6, 2022.
Application No. 17/903,507 is a continuation of application No. 17/680,718, filed on Feb. 25, 2022, now abandoned.
Application No. 17/680,718 is a continuation of application No. 17/392,158, filed on Aug. 2, 2021, now abandoned.
Application No. 17/392,158 is a continuation of application No. 17/154,498, filed on Jan. 21, 2021, now abandoned.
Application No. 17/154,498 is a continuation of application No. 15/551,093, filed on Aug. 15, 2017, now abandoned.
(60) Provisional application No. 62/117,283, filed on Feb. 17, 2015.

(51) Int. Cl. A61K 31/69; C07K 16/28; A61P 35/00; A61K 45/06; A61K 39/395

(52) U.S. Cl. CPC A61K 31/69; A61K 39/3955; A61K 45/06; A61P 35/00; C07K 16/2896; A61K 2039/545; C07K 2317/21; C07K 2317/73

(57) ABSTRACT

The present disclosure relates to methods for treating cancer, or preventing cancer recurrence or progression, comprising administering a patient an anti-CD38 antibody and a proteasome inhibitor.

### PRIORITY

This application claims priority from U.S. Provisional Pat. Application Serial No. 62/117,283, filed on Feb. 17, 2015. The entire contents of the aforementioned application are incorporated herein.

### FIELD

The present disclosure relates to the treatment of cancer using a combination therapy comprising an antibody that binds to CD38 and a proteasome inhibitor.

### SEQUENCE LISTING

This application incorporates in its entirety the Sequence Listing XML entitled “mpi15-004_seqlist.xml” (16 KB), which was last modified on Mar. 13, 2023, and filed electronically herewith.

### BACKGROUND

Multiple myeloma, a B-cell tumor of malignant plasma cells within the bone marrow, remains incurable despite advances in novel therapies with proteasome inhibitors (PIs), immunomodulating drugs (IMiD), and stem cell transplant (SCT) therapy. Multiple myeloma is characterized by the accumulation of plasma cells in the bone marrow (and other organs) and can result in bone marrow failure, bone destruction, hypercalcemia, and renal failure. It constitutes approximately 1% of all reported neoplasms and approximately 13% of hematologic cancers worldwide. In the Americas, Canada, and Western European countries, approximately 5 to 7 new cases of multiple myeloma are diagnosed per 100,000 people each year. Palumbo and Anderson, N Engl J Med 2011;364(11):1046-60; Landgren and Weiss, Leukemia 2009;23(10):1691-7; Harousseau, et al., Annals of Oncology 2008;19 Suppl 2:ii55-7. Although less common in Asian countries, incidences of multiple myeloma have increased almost 4-fold in the past 25 years and are characterized by younger age of onset, more invasive disease, and a less favorable prognosis (Huang, et al., Cancer 2007;110(4):896-905; Qiu, et al., Clinical Epidemiological Study on Multiple Myeloma in China (ASH Annual Meeting Abstracts) 2008;112(11):abstr 2723).

Multiple myeloma is sensitive to many cytotoxic drugs including alkylating agents, anthracyclines, and corticosteroids for both initial treatment and relapsed disease. Over the past decade, significant achievements have been made in expanding treatment options for multiple myeloma with novel therapies such as thalidomide, bortezomib, and lenalidomide.

Despite more therapeutic options, multiple myeloma remains incurable, and patients with early stage cancer remain at risk for relapse after their initial therapy. When patients relapse after their initial therapy, they demonstrate variable responses to subsequent treatments with decreasing likelihood and duration of response (DOR). Patients become refractory to approved therapies and ultimately are left with no alternative treatment options. Thus, there is a need for improved methods for treating such forms of cancer.

### DESCRIPTION

The present disclosure provides methods for treating cancer, or preventing cancer recurrence or progression. The methods comprise administering to a patient in need thereof i) a proteasome inhibitor of formula (I), or a pharmaceutically acceptable salt thereof, and ii) an anti-CD38 antibody.

The present disclosure further provides a use of a proteasome inhibitor of formula (I) or a pharmaceutically acceptable salt thereof, wherein the proteasome inhibitor of formula (I) or a pharmaceutically acceptable salt thereof is administered with an anti-CD38 antibody for treating cancer in a patient in need thereof.

The present disclosure further provides a use of a proteasome inhibitor of formula (I) or a pharmaceutically acceptable salt thereof in the manufacture of at least one medicament for treating cancer, wherein the proteasome inhibitor of formula (I) or a pharmaceutically acceptable salt thereof is administered with an anti-CD38 antibody to a patient in need thereof.

The present disclosure further provides a therapeutic combination comprising a compound of formula (I), or a pharmaceutically acceptable salt thereof, and an anti-CD38 antibody.

The present disclosure further provides a pharmaceutical combination comprising a composition comprising a compound of formula (I), or a pharmaceutically acceptable salt thereof, and a composition comprising an anti-CD38 antibody.

The present disclosure further provides a kit comprising an article for sale containing a combination comprising a compound of formula (I), or a pharmaceutically acceptable salt thereof, and an anti-CD38 antibody, each separately packaged with instructions for use to treat cancer.

In certain embodiments, the anti-CD38 antibody used in this invention is daratumumab, described herein.

In certain embodiments, the proteasome inhibitor of formula (I) of this disclosure is a compound of formula (IV)

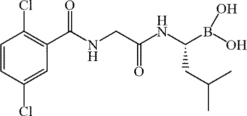 [SMILES: CC(C)C[C@H](NC(=O)CNC(=O)c1cc(Cl)ccc1Cl)B(O)O]

its esters, or a pharmaceutically acceptable salt thereof.

In certain embodiments, the proteasome inhibitor of formula (I) of this disclosure is a compound of formula (IIIa)

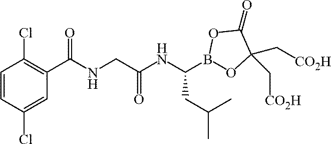 [SMILES: CC(C)C[C@H](NC(=O)CNC(=O)c1cc(Cl)ccc1Cl)B1OC(=O)C(CC(=O)O)(CC(=O)O)O1]

or a pharmaceutically acceptable salt thereof.

Unless defined otherwise, all technical and scientific terms used herein have the same meaning as commonly understood by one of ordinary skill in the art to which this present disclosure belongs. Accordingly, the following terms are intended to have the following meanings:

The term “CD38” includes any variants, isoforms and species homologs of human CD38, which are naturally expressed by cells or are expressed on cells transfected with the CD38 gene. Synonyms of CD38, as recognized in the art, include ADP ribosyl cyclase 1, cADPr hydrolase 1, Cd38-rsl, Cyclic ADP-ribose hydrolase 1, 1-19, NIM-R5 antigen. Human CD38 comprises an amino acid sequence as set forth in SEQ ID NO: 15.

The term “anti-CD38 antibody” when used herein refers to an antibody which upon binding to CD38 does not induce significant proliferation of peripheral blood mononuclear cells when compared to the proliferation induced by an isotype control antibody or medium alone (as assayed e.g. as described in Ausiello et al., Tissue Antigens 2000, 56, 539-547). In certain embodiments, an anti-CD38 antibody used in the present disclosure is not only a non-agonist, but even an antagonist of CD38.

An anti-CD38 antibody may bind to an immunoglobulin molecule such as polyclonal antibodies, monoclonal antibodies (mAbs), antibody-like polypeptides, a fragment of an immunoglobulin molecule, or a derivative of either thereof, which has the ability to specifically bind to CD38 under typical physiological conditions for significant periods of time such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to CD38).

It has been shown that the antigen-binding function of an antibody can be performed by fragments of a full-length antibody. Examples of binding fragments encompassed within the term “anti-CD38 antibody” include (i) a Fab fragment, a monovalent fragment consisting of the V_{L}, V_{H}, C_{L} and C_{H}1 domains; (ii) F(ab)₂ and F(ab′)2 fragments, bivalent fragments comprising two Fab fragments linked by a disulfide bridge at the hinge region; (iii) a Fd fragment consisting essentially of the V_{H} and C_{H}1 domains; (iv) a Fv fragment consisting essentially of the V_{L} and V_{H} domains of a single arm of an antibody, (v) a dAb fragment (Ward et al., Nature 1989, 341, 544-546), which consists essentially of a V_{H} domain; (vi) an isolated complementarity determining region (CDR), and (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain antibodies or single chain Fv (scFv) (see for instance Bird et al., Science 1988, 242, 423-426 and Huston et al., Proc. Natl. Acad. Sci. USA 1988, 85, 5879-5883). Such single chain antibodies are encompassed within the term “anti-CD38 antibody” unless otherwise noted or clearly indicated by context. Other forms of single chain antibodies, such as diabodies, are included within the term anti-CD38 antibody (see for instance Proc. Natl. Acad. Sci. USA 1993, 90(14), 6444-6448 for a description of diabodies). Although such fragments are generally included within the meaning of antibody, they collectively and each independently are unique features of the present disclosure, exhibiting different biological properties and utility. These and other useful antibody fragments in the context of the present disclosure are discussed further herein.

Various approaches to target CD38 are disclosed in the art. For example antibodies specific for CD38 are described in WO 1999/062526, US 20010031261, US 20040141982, WO 2002/006347, US 20030211553, US 2002164788, each of which is incorporated by reference in its entirety; WO 2005/103083, U.S. Pat. No. 8263746, each of which is incorporated by reference in its entirety; WO 2006/125640, US 20090123950, each of which is incorporated by reference in its entirety; WO 2007/042309, U.S. Pat. No. 8088896, each of which is incorporated by reference in its entirety; WO 2006/099875, U.S. Pat. No. 7829673, each of which is incorporated by reference in its entirety; and WO 2008/047242, U.S. Pat. No. 8153765, each of which is incorporated by reference in its entirety.

The term “daratumumab” refers to a full-length human monoclonal anti-CD38 antibody described in U.S. Pat. No. 7,829,673. Daratumumab is characterized in U.S. Pat. No. 7,829,673 as antibody -005. Daratumumab may also be referred to for example as “HuMax®-CD38.” The method to generate, isolate, and obtain daratumumab and its amino acid and encoding nucleotide sequences are described in U.S. Pat. No. 7,829,673, which is incorporated by reference specifically and in its entirety. Daratumumab comprises a heavy chain variable region comprising the amino acid sequence set forth in SEQ ID NO: 9 or encoded by the nucleotide sequence set forth in SEQ ID NO: 8. Daratumumab comprises a light chain variable region comprising the amino acid sequence set forth in SEQ ID NO: 4 or encoded by the nucleotide sequence set forth in SEQ ID NO: 3.

Proteasome inhibitors are agents that block the action of proteasomes, cellular complexes that break down proteins such as the p53 protein. Proteasome inhibitors are being studied in the treatment of cancer, especially multiple myeloma. Examples of proteasome inhibitors are: bortezomib, carfilzomib, disulfiram, epigallocatechin-3-gallate, salinosporamid A, ONX0912, CEP-18770, and Epoxomicin.

The term “orally” refers to administering a composition that is intended to be ingested. Examples of oral forms include, but are not limited to, tablets, pills, capsules, powders, granules, solutions or suspensions, and drops. Such forms may be swallowed whole or may be in chewable form.

The term “infusion” refers to the administration of a composition through a needle or catheter. Infusion may mean that a drug is administered intravenously, but the term also may refer to situations where drugs are provided through other non-oral routes, such as intramuscular injections and epidural routes (into the membranes surrounding the spinal cord).

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “comprises” refers to “includes, but is not limited to.”

The terms “boronate ester” and “boronic ester” are used interchangeably and refer to a chemical compound containing a —B(Z¹)(Z²) moiety, wherein Z¹ and Z² together form a cyclic boronic ester having 2-20 carbon atoms, and optionally one or more heteroatoms selected from N, S, or O.

The present disclosure provides methods for treating cancer, or preventing cancer recurrence or progression, in a patient in need of treatment or prevention. The methods comprise administering to a patient in need thereof i) a proteasome inhibitor of formula (I), or a pharmaceutically acceptable salt thereof, and ii) an anti-CD38 antibody.

The present disclosure further provides a therapeutic combination comprising a compound of formula (I), or a pharmaceutically acceptable salt thereof, and an anti-CD38 antibody.

The present disclosure further provides a pharmaceutical combination comprising a composition comprising a compound of formula (I), or a pharmaceutically acceptable salt thereof, and a composition comprising an anti-CD38 antibody.

The present disclosure further provides a kit comprising an article for sale containing a combination comprising a compound of formula (I), or a pharmaceutically acceptable salt thereof, and an anti-CD38 antibody, each separately packaged with instructions for use to treat cancer.

The term “patient” refers to mammalian patients, for example human patients. Patients in need of therapeutic treatment and/or prevention also include companion animals such as dogs, rats and horses.

In certain embodiments, proteasome inhibitor formula (I) refers to the following formula:

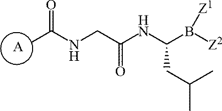

or a pharmaceutically acceptable salt, stereoisomeric or tautomeric form thereof, wherein: ring A is selected from

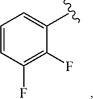

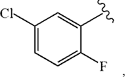

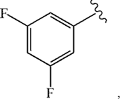

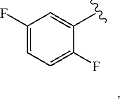

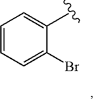

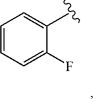

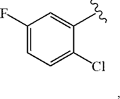

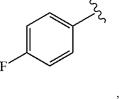

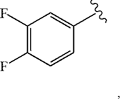

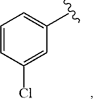

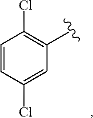

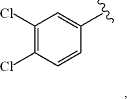

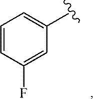

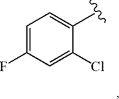

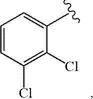

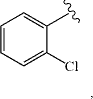

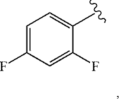

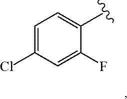

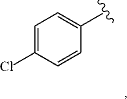

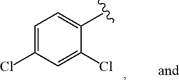

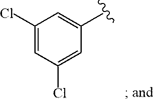

Z¹ and Z² are each independently hydroxyl; or Z¹ and Z² together form a cyclic boronic ester having 2-20 carbon atoms, and optionally one or more heteroatoms selected from N, S, or O.

In certain embodiments, Z¹ and Z² of formula (I) are each independently hydroxyl.

In certain embodiments, proteasome inhibitor formula (I) is characterized by formula (Ia):

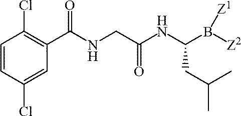

or a pharmaceutically acceptable salt, stereoisomeric or tautomeric form thereof, wherein: Z¹ and Z² are each independently hydroxyl; or Z¹ and Z² together form a cyclic boronic ester having 2-20 carbon atoms, and optionally one or more heteroatoms selected from N, S, or O.

In certain embodiments, proteasome inhibitor formula (I) is characterized by formula (II):

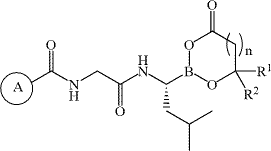

or a pharmaceutically acceptable salt, stereoisomeric or tautomeric form thereof, wherein:
ring A is defined above; R¹ and R² are each independently —(CH₂)ₚ—CO₂H; wherein one of carboxylic acids optionally forms a further bond with the boron atom;
n is 0 or 1; and p is 0 or 1.

In certain embodiments, proteasome inhibitor formula (I) is characterized by formula (III):

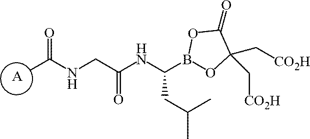

or a pharmaceutically acceptable salt, stereoisomeric or tautomeric form thereof, wherein ring A is defined above.

In certain embodiments, proteasome inhibitor formula (I) is a compound of formula (IIIa):

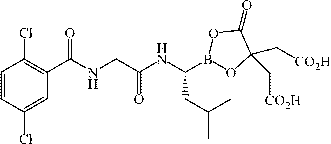 [SMILES: CC(C)C[C@H](NC(=O)CNC(=O)c1cc(Cl)ccc1Cl)B1OC(=O)C(CC(=O)O)(CC(=O)O)O1]

or a pharmaceutically acceptable salt, stereoisomeric or tautomeric form thereof.

In certain embodiments, proteasome inhibitor formula (I) is a compound of formula (IV):

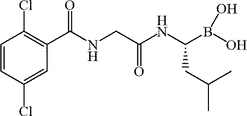 [SMILES: CC(C)C[C@H](NC(=O)CNC(=O)c1cc(Cl)ccc1Cl)B(O)O]

or a pharmaceutically acceptable salt thereof.

Synthetic methods for the preparation of proteasome inhibitors of formulas (I), (II), (III), (IIIa) and (IV) as well as pharmaceutical compositions thereof are known, for example, described in U.S. Pat. No. 7,442,830, U.S. Pat. No. 7,687,662, U.S. Pat. No. 8,003,819, U.S. Pat. No. 8,530,694, and International Patent Publication WO 2009/154737, which are hereby incorporated by reference specifically and in their entirety.

In certain embodiments, the anti-CD38 antibody is a monoclonal antibody.

In certain embodiments, the anti-CD38 antibody is a human monoclonal antibody.

In certain embodiments, the anti-CD38 antibody is an antagonist of CD38.

In certain embodiments, the anti-CD38 antibody is an isolated full-length antibody that binds to human CD38.

The methods to generate, isolate, and obtain anti-CD38 antibodies are well known in the art, for example, described in U.S. Pat. No. 7,829,673, U.S. Pat. Publication No. 2010/0092489, U.S. Pat. Publication No. 2013/0209355, which are hereby incorporated by reference specifically and in their entirety.

In certain embodiments, the anti-CD38 antibody is an antibody comprising a V_{L} region having the amino acid sequence as set forth in SEQ ID NO: 4.

In certain embodiments, the anti-CD38 antibody is an antibody comprising a V_{H} region having the amino acid sequence as set forth in SEQ ID NO: 9.

In certain embodiments, the anti-CD38 antibody comprises a V_{L} region having the amino acid sequence as set forth in SEQ ID NO: 4 and a V_{H} region having the amino acid sequence as set forth in SEQ ID NO: 9.

In certain embodiments, the anti-CD38 antibody is an antibody comprising a V_{L} CDR1 having the amino acid sequence as set forth in SEQ ID NO: 5.

In certain embodiments, the anti-CD38 antibody is an antibody comprising a V_{L} CDR2 having the amino acid sequence as set forth in SEQ ID NO: 6.

In certain embodiments, the anti-CD38 antibody is an antibody comprising a V_{L} CDR3 having the amino acid sequence as set forth in SEQ ID NO: 7.

In certain embodiments, the anti-CD38 antibody is an antibody comprising a V_{H} CDR1 having the amino acid sequence as set forth in SEQ ID NO: 10.

In certain embodiments, the anti-CD38 antibody is an antibody comprising a V_{H} CDR2 having the amino acid sequence as set forth in SEQ ID NO: 11.

In certain embodiments, the anti-CD38 antibody is an antibody comprising a V_{H} CDR3 having the amino acid sequence as set forth in SEQ ID NO: 12.

In certain embodiments, the anti-CD38 antibody comprises a V_{L} CDR1 region comprising the amino acid sequence as set forth in SEQ ID NO: 5, a V_{L} CDR2 region comprising the amino acid sequence as set forth in SEQ ID NO: 6, a V_{L} CDR3 region comprising the amino acid sequence as set forth in SEQ ID NO: 7, a V_{H} CDR1 region comprising the amino acid sequence as set forth in SEQ ID NO: 10, a V_{H} CDR2 region comprising the amino acid sequence as set forth in SEQ ID NO: 11, and a V_{H} CDR3 region comprising the amino acid sequence as set forth in SEQ ID NO: 12.

In certain embodiments, the anti-CD38 antibody is daratumumab.

The methods to generate, isolate, and obtain anti-CD38 antibodies are well known in the art, for example, described in U.S. Pat. No. 7,829,673, U.S. Pat. Publication No. 2010/0092489, U.S. Pat. Publication No. 2013/0209355, which are hereby incorporated by reference specifically and in their entirety.

In certain embodiments, the anti-CD38 antibody is an antibody comprising a V_{H} region having the amino acid sequence as set forth in SEQ ID NO: 2.

In certain embodiments, the anti-CD38 antibody is an antibody comprising a V_{L} region having the amino acid sequence as set forth in SEQ ID NO: 1.

In certain embodiments, the anti-CD38 antibody is an antibody comprising a V_{H} region having the amino acid sequence as set forth in SEQ ID NO: 2 and a V_{L} region having the amino acid sequence as set forth in SEQ ID NO: 1.

In certain embodiments, the anti-CD38 antibody is an antibody comprising a V_{H} region having the amino acid sequence as set forth in SEQ ID NO: 14.

In certain embodiments, the anti-CD38 antibody is an antibody comprising a V_{L} region having the amino acid sequence as set forth in SEQ ID NO: 13.

In certain embodiments, the anti-CD38 antibody is an antibody comprising a V_{H} region having the amino acid sequence as set forth in SEQ ID NO: 14 and a V_{L} region having the amino acid sequence as set forth in SEQ ID NO: 13.

In certain embodiments, the proteasome inhibitor is a compound of formula (IIIa) and the anti-CD38 antibody comprises a V_{L} region having the amino acid sequence as set forth in SEQ ID NO: 4 and a V_{H} region having the amino acid sequence as set forth in SEQ ID NO: 9.

The methods to generate, isolate, and obtain anti-CD38 antibodies are well known in the art, for example, described in U.S. Pat. No. 7,829,673, U.S. Pat. Publication No. 2010/0092489, U.S. Pat. Publication No. 2013/0209355, which are hereby incorporated by reference specifically and in their entirety.

In certain embodiments, a therapeutic combination comprising a proteasome inhibitor and an anti-CD38 antibody is administered with one or more therapeutic agents. The other therapeutic agents may also inhibit the proteasome, or may operate by a different mechanism. In certain embodiments, the other therapeutic agents are those that are normally administered to patients with the disease or condition being treated. The other therapeutic agent(s) may be administered with the proteasome inhibitor or anti-CD38 antibody in a single dosage form or as a separate dosage form. When administered as a separate dosage form, the other therapeutic agent(s) may be administered prior to, at the same time as, or following administration of proteasome inhibitor or anti-CD38 antibody.

In certain embodiments, a therapeutic combination comprising a proteasome inhibitor and an anti-CD38 antibody are administered with one or more anticancer agent(s). As used herein, the term “anticancer agent” refers to any agent that is administered to a patient with cancer for purposes of treating the cancer.

In certain embodiments, the other therapeutic agent includes DNA damaging chemotherapeutic agents such as topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

In certain embodiments, the other therapeutic agent includes chemotherapeutic agents that disrupt cell replication such as: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which downregulates cell replication.

In certain embodiments, the proteasome inhibitor of formula (I) and anti-CD38 antibody are administered with an immunomodulating agent. In such embodiments, the immunomodulating agent is thalidomide, lenalidomide or pomalidomide. In certain embodiments, the proteasome inhibitor of formula (I) and anti-CD38 antibody are administered with lenalidomide.

In certain embodiments, the proteasome inhibitor of formula (I) and anti-CD38 antibody are administered with an alkylating agent. In such embodiments, the alkylating agent is melphalan or cyclophosphamide. In certain embodiments, the proteasome inhibitor of formula (I) and anti-CD38 antibody are administered with melphalan. In certain embodiments, the proteasome inhibitor of formula (I) and anti-CD38 antibody are administered with cyclophosphamide.

In certain embodiments, the proteasome inhibitor of formula (I) and anti-CD38 antibody are administered with a steroid. In such embodiments, the steroid is dexamethasone, prednisone, prednisolone, or methylprednisone. In certain embodiments, the proteasome inhibitor of formula (I) and anti-CD38 antibody are administered with dexamethasone.

In certain embodiments, the proteasome inhibitor of formula (I) and anti-CD38 antibody are administered with cyclophosphamide and dexamethasone.

In certain embodiments, the proteasome inhibitor of formula (IIIa) or (IV) and daratumumab are administered with lenalidomide.

In certain embodiments, the proteasome inhibitor of formula (IIIa) or (IV) and daratumumab are administered with melphalan.

In certain embodiments, the proteasome inhibitor of formula (IIIa) or (IV) and daratumumab are administered with cyclophosphamide.

In certain embodiments, the proteasome inhibitor of formula (IIIa) or (IV) and daratumumab are administered with dexamethasone.

In certain embodiments, the proteasome inhibitor of formula (IIIa) or (IV) and daratumumab are administered with cyclophosphamide and dexamethasone.

In certain embodiments, the method of this disclosure further comprises administering to a patient melphalan, lenalidomide, cyclophosphamide, and/or dexamethasone.

The proteasome inhibitors or anti-CD38 antibodies used in the present disclosure may be formulated as a pharmaceutical composition with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, PA, 1995.

Pharmaceutical compositions used in the present disclosure may also include diluents, fillers, salts, buffers, detergents (e. g., a nonionic detergent, such as Tween- 80), stabilizers (e. g., sugars or protein-free amino acids), preservatives, tissue fixatives, solubilizers, and/or other materials suitable for inclusion in a pharmaceutical composition.

The compounds used in the present disclosure may be administered via any suitable route, such as an oral, nasal, inhalable, topical (including buccal, transdermal and sublingual), rectal, vaginal and/or parenteral route.

In certain embodiments, one or more of the proteasome inhibitors used in the present disclosure are administered orally, for example, with an inert diluent or an assimilable edible carrier. The active ingredient may be enclosed in a hard or soft shell gelatin capsule, or compressed into tablets. Pharmaceutical compositions which are suitable for oral administration include ingestible tablets, buccal tablets, troches, capsules, elixirs, suspensions, syrups, wafers, and the like containing such carriers as are known in the art to be appropriate.

In certain embodiments, one or more of the antibodies used in the present disclosure are administered parenterally. The phrases “parenteral administration” and “administered parenterally” as used herein mean modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

In certain embodiments of the methods of the disclosure, the proteasome inhibitor is a compound of formula (IIIa), wherein the compound of formula (IIIa) is administered orally.

In certain embodiments of the methods of the disclosure, the proteasome inhibitor is a compound of formula (IIIa), wherein the compound of formula (IIIa) is enclosed in a capsule and administered orally.

In certain embodiments of the methods of the disclosure, the anti-CD38 antibody is administered by infusion.

In certain embodiments of the methods of the disclosure, the anti-CD38 antibody is daratumumab, wherein daratumumab is administered by infusion.

In certain embodiments of the methods of the disclosure, the anti-CD38 antibody is daratumumab, wherein daratumumab is administered by intravenous infusion.

In certain embodiments of the methods of the disclosure, the anti-CD38 antibody is daratumumab, wherein daratumumab is administered by continuous infusion over a period of from 2 to 24 hours.

In certain embodiments of the methods of the disclosure, the anti-CD38 antibody is daratumumab, wherein daratumumab is administered by continuous infusion over a period of from 2 to 12 hours.

In certain embodiments of the methods of the disclosure, the anti-CD38 antibody is administered by subcutaneous infusion.

In certain embodiments of the methods of the disclosure, the anti-CD38 antibody is daratumumab, wherein daratumumab is administered by subcutaneous infusion.

In certain embodiments of the methods of the disclosure, the anti-CD38 antibody is daratumumab, wherein daratumumab is administered with recombinant human hyaluronidase enzyme (rHuPH20).

In certain embodiments of the methods of the disclosure, the anti-CD38 antibody is daratumumab, wherein daratumumab is administered by subcutaneous infusion over a period of less than 2 hours.

In certain embodiments of the methods of the disclosure, the proteasome inhibitor is a compound of formula (IIIa) and the anti-CD38 antibody is daratumumab, wherein the compound of formula (IIIa) is enclosed in a capsule and administered orally and daratumumab is administered by infusion.

The methods of this disclosure are useful for treating a patient having, or at risk of developing or experiencing a recurrence of, a proteasome-mediated disorder.

As used herein, the term “proteasome-mediated disorder” includes any disorder, disease or condition which is caused or characterized by an increase in proteasome expression or activity. The term “proteasome-mediated disorder” also includes any disorder, disease or condition in which inhibition of proteasome activity is beneficial.

For example, the methods of this disclosure are useful in treatment of disorders mediated via proteins (e.g., NFκB, p27^{Kip}, p21^{WAF/CIP1}, p53) which are regulated by proteasome activity. Exemplary proteasome-mediated disorders include inflammatory disorders (e.g., rheumatoid arthritis, inflammatory bowel disease, asthma, chronic obstructive pulmonary disease (COPD), osteoarthritis, dermatosis (e.g., atopic dermatitis, psoriasis)), vascular proliferative disorders (e.g., atherosclerosis, restenosis), proliferative ocular disorders (e.g., diabetic retinopathy), benign proliferative disorders (e.g., hemangiomas), autoimmune diseases (e.g., multiple sclerosis, tissue and organ rejection), as well as inflammation associated with infection (e.g., immune responses), antibody-mediated disease, neurodegenerative disorders (e.g., Alzheimer’s disease, Parkinson’s disease, Amyotrophic Lateral Sclerosis, motor neurone disease, neuropathic pain, triplet repeat disorders, astrocytoma, and neurodegeneration as result of alcoholic liver disease), ischemic injury (e.g., stroke), and cachexia including accelerated muscle protein breakdown that accompanies various physiological and pathological states (e.g., nerve injury, fasting, fever, acidosis, HIV infection, cancer affliction, and certain endocrinopathies), or useful for desensitization therapy.

Non-limiting examples of autoimmune diseases and antibody-mediated diseases include systemic lupus erythematosus, lupus nephritis, Sjogren’s syndrome, ulcerative colitis, Crohn’s disease, type 1 diabetes, myasthenia gravis, idiopathic pulmonary fibrosis, cirrhosis, endomyocardial fibrosis, scleroderma sclerosis, systemic sclerosis, antibody-mediated rejection, antibody-mediated rejection in organ transplantation, antibody-mediated rejection in kidney transplantation, antibody-mediated rejection in lung transplantation, antibody-mediated rejection in heart transplantation, antibody-mediated rejection in liver transplantation, antibody-mediated rejection in pancreas transplantation, or graft versus host disease.

The methods of this disclosure provide efficacious treatments for patients with cancer. As used herein, the term “cancer” refers to a cellular disorder characterized by uncontrolled or dis-regulated cell proliferation, decreased cellular differentiation, inappropriate ability to invade surrounding tissue, and/or ability to establish new growth at ectopic sites. The term “cancer” includes, but is not limited to, solid tumors and hematologic malignancies. The term “cancer” encompasses diseases of skin, tissues, organs, bone, cartilage, blood, and vessels. The term “cancer” further encompasses primary and metastatic cancers.

Non-limiting examples of solid tumors that can be treated with the methods of this disclosure include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

Non-limiting examples of hematologic malignancies that can be treated with the methods of this disclosure include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin’s disease (HD); lymphoma; non-Hodgkin’s lymphoma (NHL), including follicular lymphoma and mantle cell lymphoma; B-cell lymphoma; T-cell lymphoma; multiple myeloma (MM); Waldenstrom’s macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

In certain embodiments, the methods of this disclosure are useful in treatment of amyloidosis.

In certain embodiments, the methods of this disclosure are used to treat a patient having or at risk of developing or experiencing a recurrence (relapse) in a cancer selected from multiple myeloma and mantle cell lymphoma.

In certain embodiments, the methods of this disclosure are used to treat a patient with refractory mantle cell lymphoma.

In certain embodiments, the methods of this disclosure are used to treat a patient with multiple myeloma.

In certain embodiments, the methods of this disclosure are used to treat a patient with refractory multiple myeloma.

In certain embodiments, the anti-CD38 antibody is administered to a patient in need thereof in a dose of from about 1-100 mg/kg.

In certain embodiments, the anti-CD38 antibody is administered to a patient in need thereof in a dose of from about 2-50 mg/kg.

In certain embodiments, the anti-CD38 antibody is administered to a patient in need thereof in a dose of from about 2-40 mg/kg.

In certain embodiments, the anti-CD38 antibody is administered to a patient in need thereof in a dose of from about 2-30 mg/kg.

In certain embodiments, the anti-CD38 antibody is administered to a patient in need thereof in a dose of from about 4-20 mg/kg.

In certain embodiments, the anti-CD38 antibody is administered to a patient in need thereof in a dose of about 8 mg/kg.

In certain embodiments, the anti-CD38 antibody is administered to a patient in need thereof in a dose of about 16 mg/kg.

In certain embodiments, the anti-CD38 antibody is administered daily, once every two days, once every three days, once every four days, once every five days, once every six days, once a week, once every two weeks, or once every four weeks.

In certain embodiments, the anti-CD38 antibody is administered once weekly for 2 to 20 weeks, such as for 3 to 12 weeks, such as for 4 to 8 weeks.

In certain embodiments, the anti-CD38 antibody is administered once every two weeks, for a period of 1 month or more.

In certain embodiments, the anti-CD38 antibody is administered once every four weeks, for a period of 1 month or more.

In certain embodiments, the anti-CD38 antibody is administered once weekly for 2-20 weeks, then once every two weeks for a period of 1 month or more, then once every four weeks for a period of 1 month or more.

In certain embodiments, the anti-CD38 antibody is administered once weekly for 2-20 weeks, then once every two weeks for a period of 1 month or more.

In certain embodiments, the anti-CD38 antibody is administered once weekly for 2-20 weeks, then once every four weeks for a period of 1 month or more.

In certain embodiments, the anti-CD38 antibody is administered once every two weeks for a period of 1 month or more, then once every four weeks for a period of 1 month or more.

In certain embodiments, the anti-CD38 antibody is administered once weekly for weeks 1-8 weeks, then once every two weeks for weeks 9-24 then once every four weeks for weeks 25 until disease progression.

In certain embodiments, the anti-CD38 antibody is administered once weekly for a 28 day cycle repeated twice, then every two week in cycles 3-6 and then every 4 weeks in subsequent cycles until disease progression.

In certain embodiments, the anti-CD38 antibody is administered by infusion in a dosage of from 10 to 500 mg/m², such as of from 200 to 400 mg/m². Such administration may be repeated, e.g., 1 to 8 times, such as 3 to 5 times. The administration may be performed by continuous infusion over a period of from 2 to 24 hours, such as of from 2 to 12 hours.

In certain embodiments, the anti-CD38 antibody is administered by slow continuous infusion over a long period, such as more than 24 hours, in order to reduce toxic side effects. In certain embodiments the anti-CD38 antibody is administered once a week, once every two weeks, or once every four weeks in a dosage of from 250 mg/m² to 2000 mg/m², such as for example 300 mg/m², 500 mg/m², 700 mg/m², 1000 mg/m², 1500 mg/m² or 2000 mg/m², for up to 8 times, such as from 4 to 6 times. The administration may be performed by continuous infusion over a period of from 2 to 24 hours, such as of from 2 to 12 hours. Such regimen may be repeated one or more times as necessary, for example, after 6 months or 12 months. The dosage may be determined or adjusted by measuring the amount of compound of the present disclosure in the blood upon administration by for instance taking out a biological sample and using anti-idiotypic antibodies which target the antigen binding region of the anti-CD38 antibody.

In certain embodiments, the proteasome inhibitor is administered to a patient in need thereof in a dose of from about 0.5-20 mg.

In certain embodiments, the proteasome inhibitor is administered to a patient in need thereof in a dose of from about 1-12 mg.

In certain embodiments, the proteasome inhibitor is administered to a patient in need thereof in a dose of from about 1.5-10 mg.

In certain embodiments, the proteasome inhibitor is administered to a patient in need thereof in a dose of about 2.3 mg.

In certain embodiments, the proteasome inhibitor is administered to a patient in need thereof in a dose of about 3.0 mg.

In certain embodiments, the proteasome inhibitor is administered to a patient in need thereof in a dose of about 4.0 mg.

In certain embodiments, the proteasome inhibitor is administered to a patient in need thereof in a dose of about 5.3 mg.

In certain embodiments, the proteasome inhibitor is administered to a patient in need thereof in a dose of about 5.5 mg.

In certain embodiments, the proteasome inhibitor is administered daily, once every two days, once every three days, once every four days, once every five days, once every six days, weekly, once every two weeks, or once every four weeks.

In certain embodiments, the proteasome inhibitor is administered once weekly for 2-20 weeks.

In certain embodiments, the proteasome inhibitor is administered on days 1, 8, 15 of a 28-day schedule.

In certain embodiments, the proteasome inhibitor is administered daily, once every two days, once every three days, once every four days, once every five days, once every six days, weekly, once every two weeks, or once every four weeks, and the anti-CD38 antibody is administered daily, once every two days, once every three days, once every four days, once every five days, once every six days, once a week, once every two weeks, or once every four weeks.

In certain embodiments, patients are administered with proteasome inhibitor of formula (I) in a dosage of 0.5-20 mg and with anti-CD38 antibody in a dosage of 1-100 mg/kg.

In certain embodiments, patients are administered with proteasome inhibitor of formula (I) in a dosage of 1-12 mg and with anti-CD38 antibody in a dosage of 2-50 mg/kg.

In certain embodiments, patients are administered with proteasome inhibitor of formula (IIIa) in a dosage of 1-12 mg and with daratumumab in a dosage of 2-50 mg/kg.

In certain embodiments, patients are administered with the proteasome inhibitor of (IIIa) in a dosage of 2.3, 3, 4 or 5.5 mg per scheduled dose and with daratumummab in a dosage of 8 or 16 mg/kg per scheduled dose.

In certain embodiments, the proteasome inhibitor of formula (I) and anti-CD38 antibody may be administered simultaneously or sequentially in any order. In certain embodiments, they may be administered separately or in one or more pharmaceutical compositions.

In certain embodiments, a given dosing schedule comprises one or more administrations of a proteasome inhibitor/anti-CD38 antibody, wherein at least one administration of a proteasome inhibitor/anti-CD38 antibody, such as described herein, may be repeated or cycled on a daily, weekly, biweekly, monthly, bimonthly, annually, semi-annually, or any other period. A repeated dosing schedule or cycle may be repeated for a fixed period of time determined at the start of the schedule; may be terminated, extended, or otherwise adjusted based on a measure of therapeutic effect, such as a level of reduction in the presence of detectable disease tissue (e.g. a reduction of at least 50%, 60%, 70%, 80%, 90%, 95%, 99%, or 100%); or may be terminated, extended, or otherwise adjusted for any other reason as determined by a medical professional.

In certain embodiments, the dosing regimen is an intermittent regimen. In certain embodiments, the intermittent regimen comprises at least one cycle of a treatment period of at least 1 day followed by a rest period of at least 1 day. In certain embodiments, the intermittent dosing regimen can comprise at least one cycle of a treatment period of 2, 3, 4, 5, 6 or 7 days followed by a rest period of at least 1 day. In certain embodiments, the intermittent dosing regimen comprises at least one cycle of a treatment period of 2, 3, 4, 5, 6 or 7 days followed by a rest period of at least 3, 4, or 5 days, at least one cycle of a treatment period of at least 1 day followed by a rest period of 6 days, at least one 7-day cycle of a treatment period of 3 days followed by a rest period of 4 days, at least one 7-day cycle of a treatment period of 5 days followed by a rest period of 2 days, or at least one 7-day cycle of a treatment period of 1 day followed by a rest period of 6 days. In certain embodiments, the intermittent dosing regimen can comprise at least one cycle of a treatment period of 1 day followed by a rest period of 13 days. In certain embodiments, the intermittent dosing regimen can comprise at least one cycle of a treatment period of 1 day followed by a rest period of 20 days. In certain embodiments, the intermittent dosing regimen can comprise at least one cycle of a treatment period of 1 day followed by a rest period of 27 days. In certain embodiments, the intermittent dosing regimen comprises at least one 7-day cycle comprising at least 3 treatment periods on alternate days within the 7 days.

In some combination therapy regimens, a patient is administered a combination of (a) a proteasome inhibitor according to a first dosing regimen and (b) an anti-CD38 antibody according to a second dosing regimen. The first dosing regimen and the second dosing regimen can be different, or can be the same and are administered simultaneously. Each dosing regimen independently comprises repeating cycles of a treatment period followed by a rest period. Preferably, at least one dosing regimen has one rest period of more than 0 day. In some combination regimens, one of the first and second dosing regimens is not an intermittent regimen, i.e., a continuous regimen. For example, in certain embodiments, either the first or the second regimen has a rest period of 0 day.

In certain embodiments, the first and/or the second dosing regimen independently comprises at least one cycle of a treatment period of at least 1 day followed by a rest period of at least 1 day. In certain embodiments, the first and/or the second dosing regimen can independently comprise at least one cycle of a treatment period of 2, 3, 4, 5, 6 or 7 days followed by a rest period of at least 1 day. In certain embodiments, the first and/or the second dosing regimen independently comprises at least one cycle of a treatment period of 2, 3, 4, 5, 6 or 7 days followed by a rest period of at least 3, 4, or 5 days. In certain embodiments, the first and/or the second dosing regimen independently comprises at least one cycle of a treatment period of at least 1 day followed by a rest period of 6 days, at least one cycle of a treatment period of 1 day followed by a rest period of 13 days, at least one cycle of a treatment period of 1 day followed by a rest period of 20 days, or at least one cycle of a treatment period of 1 day followed by a rest period of 27 days. In certain embodiments, the first and/or the second dosing regimen independently comprises at least one 7-day cycle of a treatment period of 3 days followed by a rest period of 4 days, or at least one 7-day cycle of a treatment period of 5 days followed by a rest period of 2 days, or at least one 7-day cycle of a treatment period of 1 day followed by a rest period of 6 days, or at least one 7-day cycle of a treatment period of 1 day followed by a rest period of 6 days. Optionally, the first dosing regimen and the second dosing regimen are the same and are administered simultaneously.

We claim:

1. A method for treating cancer, or preventing cancer recurrence or progression in a patient in need thereof, the method comprising:
administering to the patient an anti-CD38 antibody and a proteasome inhibitor of formula (I) 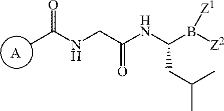
or a pharmaceutically acceptable salt thereof, wherein ring A is selected from 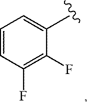 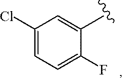 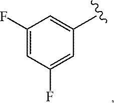 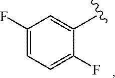 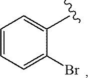 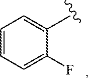 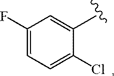 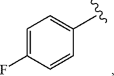 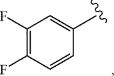 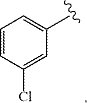 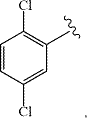 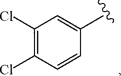 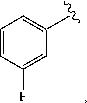 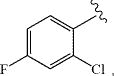 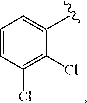 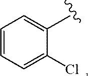 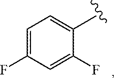 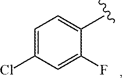 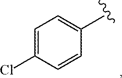 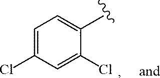 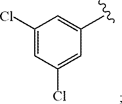
and
Z¹ and Z² are each independently hydroxyl; or Z¹ and Z² together form a cyclic boronic ester having 2-20 carbon atoms, and optionally one or more heteroatoms selected from N, S, or O.

2. The method of claim 1, wherein the proteasome inhibitor of formula (I) is a compound of formula (Ia): 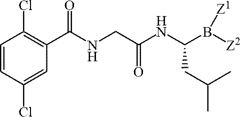
or a pharmaceutically acceptable salt thereof, wherein: Z¹ and Z² are each independently hydroxyl; or Z¹ and Z² together form a cyclic boronic ester having 2-20 carbon atoms, and optionally one or more heteroatoms selected from N, S, or O.

3. The method of claim 1, wherein the proteasome inhibitor is a compound of formula (II) 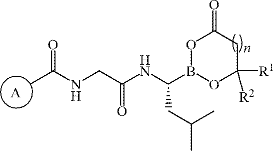
or a pharmaceutically acceptable salt thereof, wherein:
R¹ and R² are each independently —(CH₂)ₚ—CO₂H; wherein one of carboxylic acids optionally forms a further bond with the boron atom;
n is 0 or 1; and p is 0 or 1.

4. The method of claim 1, wherein the proteasome inhibitor is a compound of formula (III) 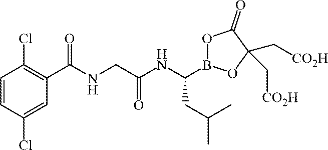 [SMILES: CC(C)C[C@H](NC(=O)CNC(=O)c1cc(Cl)ccc1Cl)B1OC(=O)C(CC(=O)O)(CC(=O)O)O1]
or a pharmaceutically acceptable salt thereof.

5. The method of claim 1, wherein the proteasome inhibitor is a compound of formula (IV) 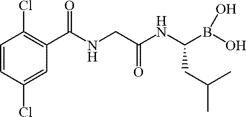 [SMILES: CC(C)C[C@H](NC(=O)CNC(=O)c1cc(Cl)ccc1Cl)B(O)O]
its esters, or a pharmaceutically acceptable salt thereof.

6. The method of claim 1, wherein said anti-CD38 antibody is a human monoclonal antibody.

7. The method of claim 6, wherein said human monoclonal antibody is a human IgG1 monoclonal antibody.

8. The method of claim 6, wherein said anti-CD38 antibody is an antagonist of CD38.

9. The method of claim 6, wherein said anti-CD38 antibody is an isolated full-length antibody that binds to human CD38.

10. The method of claim 9, wherein said anti-CD38 antibody binds to CD38 having an amino acid sequence as set forth in SEQ ID NO: 15.

11. The method of claim 6, wherein said anti-CD38 antibody comprises:
a) a V_{L} CDR1 region comprising the amino acid sequence as set forth in SEQ ID NO: 5;
b) a V_{L} CDR2 region comprising the amino acid sequence as set forth in SEQ ID NO: 6;
c) a V_{L} CDR3 region comprising the amino acid sequence as set forth in SEQ ID NO: 7;
d) a V_{H} CDR1 region comprising the amino acid sequence as set forth in SEQ ID NO: 10;
e) a V_{H} CDR2 region comprising the amino acid sequence as set forth in SEQ ID NO: 11; and
f) a V_{H} CDR3 region comprising the amino acid sequence as set forth in SEQ ID NO: 12.

12. The method of claim 6, wherein said anti-CD38 antibody comprises a V_{L} region having the amino acid sequence as set forth in SEQ ID NO: 4.

13. The method of claim 6, wherein said anti-CD38 antibody comprises a V_{H} region having the amino acid sequence as set forth in SEQ ID NO: 9.

14. The method of claim 6, wherein said anti-CD38 antibody comprises a V_{L} region having the amino acid sequence as set forth in SEQ ID NO: 4 and a V_{H} region having the amino acid sequence as set forth in SEQ ID NO: 9.

15. The method of claim 1, wherein said proteasome inhibitor is a compound of formula (IIIa) and said anti-CD38 antibody comprises a V_{L} region having the amino acid sequence as set forth in SEQ ID NO: 4 and a V_{H} region having the amino acid sequence as set forth in SEQ ID NO: 9.

16. The method of claim 1, wherein said proteasome inhibitor is a compound of formula (IIIa) and said anti-CD38 antibody comprises:
a) a V_{L} CDR1 region comprising the amino acid sequence as set forth in SEQ ID NO: 5;
b) a V_{L} CDR2 region comprising the amino acid sequence as set forth in SEQ ID NO: 6;
c) a V_{L} CDR3 region comprising the amino acid sequence as set forth in SEQ ID NO: 7;
d) a V_{H} CDR1 region comprising the amino acid sequence as set forth in SEQ ID NO: 10;
e) a V_{H} CDR2 region comprising the amino acid sequence as set forth in SEQ ID NO: 11; and
f) a V_{H} CDR3 region comprising the amino acid sequence as set forth in SEQ ID NO: 12.

17. The method of claim 1, wherein the cancer is multiple myeloma, lymphoma, refractory multiple myeloma or lymphoma, or recurrence of multiple myeloma or lymphoma.

18. The method of claim 1, wherein the proteasome inhibitor is administered with one or more therapeutic agents.

19. The method of claim 18, wherein the therapeutic agent is melphalan, lenalidomide, cyclophosphamide, or dexamethasone.

20. A therapeutic combination comprising a compound of formula (I) of claim 1, or a pharmaceutically acceptable salt thereof, and an anti-CD38 antibody.

21. A pharmaceutical combination comprising a composition comprising a compound of formula (I) of claim 1, or a pharmaceutically acceptable salt thereof, and a composition comprising an anti-CD38 antibody.
